(19) United States
(12) Patent Application Publication
(10) Pub. No.: US 2023/0052189 A1
(43) Pub. Date: Feb. 16, 2023

(54) TREATMENT OF PRIMARY BILIARY CHOLANGITIS WITH ELAFIBRANOR

(71) Applicant: Genfit, Loos (FR)

(72) Inventors: Stephanie Petit, Wattignies (FR); Julie Mouton, Baisieux (FR); Julie Dietrich, Boston, MA (US); Omar Olhaye, Derwood, MD (US); Pascal Birman, Paris (FR)

(21) Appl. No.: 17/792,217

(22) PCT Filed: Feb. 4, 2021
(86) PCT No.: PCT/EP2021/052710

§ 371 Date: Jul. 12, 2022

(30) Foreign Application Priority Data
Feb. 10, 2020 (EP) 20305124.8

(51) Int. Cl. A61K 31/192; A61K 9/00; A61K 9/28; A61P 1/16

(52) U.S. Cl. CPC A61K 31/192; A61K 9/0053; A61K 9/28; A61P 1/16

(57) ABSTRACT

The invention relates to a pharmaceutical composition comprising elafibranor, GFT1007 or a pharmaceutically acceptable salt of elafibranor or of GFT1007, for use to treat primary biliary cholangitis (PBC) without provoking and/or worsening at least one adverse event associated to PBC.

Primary biliary cholangitis (PBC) is a rare, chronic, progressive liver disease of autoimmune etiology, characterized by injury of the intrahepatic bile ducts that, in untreated patients, may progress to hepatic fibrosis, cirrhosis, hepatic decompensation, and death unless patients receive a liver transplant. PBC disproportionately affects women vs men (approximately 10:1) and is typically diagnosed in patients between 40 years to 60 years of age. In Europe, North America, Asia, and Australia, the incidence and prevalence rates of PBC are reported as ranging from 0.33 to 5.8 per 100,000 inhabitants and 1.91 to 40.2 per 100,000 inhabitants, respectively. Over 60% of the newly diagnosed cases are asymptomatic. The majority of asymptomatic patients become symptomatic within 10 years. The most common symptoms of PBC are fatigue and pruritus (Crosignani A, et al., Clinical features and management of primary biliary cirrhosis. World J Gastroenterol. 2008; 14(21):3313-3327). The mechanisms underlying these symptoms are not well elucidated and neither correlates with disease stage or clinical outcomes. PBC represents one of the leading indications for liver transplantation. Despite its rarity,

PBC remains therefore an important cause of morbidity in the Western world. PBC has also been identified as an important risk factor for hepatocellular carcinoma.

PBC is characterized by cholestasis caused by autoimmune destruction of biliary ductules with progressive impairment of bile flow in the liver. This results in increased hepatocellular bile acid concentrations which are toxic to the liver. Such hepatocellular injury is associated with a local inflammatory response resulting early on in an abnormal elevation of serum alkaline phosphatase (ALP) levels. Indeed, elevations in ALP level are associated with a risk of liver transplantation or death that is 2.0 to 2.5 times higher than the risk associated with normal levels. An abnormally elevated bilirubin level, which occurs later in disease progression, is also a strong predictor of outcomes, with a risk of liver transplantation or death that was 5.1 to 10.7 times the risk associated with normal levels.

The only approved drugs to treat patients with PBC are ursodeoxycholic acid (UDCA) and more recently Ocaliva® (obeticholic acid, OCA).

UDCA (Ursodeoxycholic acid) has been shown to improve ALP and bilirubin levels, and to delay histological progression, thereby increasing liver transplant-free survival. However, up to 40% of UDCA-treated patients have a suboptimal response (Ali A H, et al., Orphan drugs in development for primary biliary cirrhosis: challenges and progress. Orphan Drugs: Research and Reviews. 2015; 5:83 -97 ). ALP has been shown to remain elevated in up to 70% of patients who are currently being treated or are intolerant to UDCA (Lammers W J, et al., Levels of alkaline phosphatase and bilirubin are surrogate end points of outcomes of patients with primary biliary cirrhosis: an international follow-up study. Gastroenterology. 2014; 147(6):1338-1349.). Such patients remain at risk of disease progression and longer term adverse clinical outcomes. At present, 44% of UDCA-treated patients are progressing to liver transplant or death over 15 years.

Obeticholic acid (OCA), which has been shown to reduce ALP, has been recently approved in several countries as second line therapy for the treatment of PBC as monotherapy in adults unable to tolerate UDCA or in combination with UDCA in adults with an inadequate response to UDCA. A decrease in ALP levels is recognized as a particularly relevant surrogate marker for the treatment of PBC, and was recently used as the basis for conditional market approval of OCA in this indication.

However, severe pruritus has been reported with OCA (Nevens F, et al., A placebo-controlled trial of obeticholic acid in primary biliary cholangitis. N Engl J Med. 2016; 375(7):631-643) and improvement in survival or disease-related symptoms has not yet been established.

Considering the efficacy and tolerability issues with the current treatment options available, there is an unmet need for therapeutic options for patients with PBC, allowing treatment of PBC, without provoking or worsening secondary effects of the treatment.

Elafibranor (2-(2,6-dimethyl-4-{3-[4-(methylsulfanyl)phenyl]-3-oxopropen-1-yl}phenoxy)-2-methylpropanoic acid) is a drug currently tested in a pivotal phase III study for the treatment of non-alcoholic steatohepatitis. Elafibranor was also evaluated in a phase II study for the treatment of PBC. The results of phase II on PBC show that the mean relative change (%) from baseline to Endpoint in serum ALP was −48.3% for the elafibranor 80 mg treatment group, −40.6% for the elafibranor 120 mg treatment group, and 3.2% for placebo. The absolute change from baseline in serum ALP was statistically significantly different from placebo at Endpoint for both the elafibranor 80 mg treatment group (p<0.001) and the elafibranor 120 mg treatment group.

Thus, the treatment with elafibranor resulted in a consistent, statistically significant reduction in plasma ALP levels from baseline when compared to placebo. Moreover, elafibranor is safe and well-tolerated by the patients.

Thus, the invention relates to a pharmaceutical composition comprising a compound selected from elafibranor, GFT1007, a pharmaceutical salt of elafibranor and a pharmaceutically acceptable salt of GFT1007 for use to treat PBC without provoking and/or worsening at least one adverse event associated to PBC. In a particular embodiment, the adverse event is pruritus.

The invention further relates to a pharmaceutical composition comprising a compound selected from elafibranor, GFT1007, a pharmaceutical salt of elafibranor and a pharmaceutically acceptable salt of GFT1007, for use in a method for the treatment of PBC in a subject having PBC with pruritus.

The invention also relates to a pharmaceutical composition comprising a compound selected from elafibranor, GFT1007, a pharmaceutical salt of elafibranor and a pharmaceutically acceptable salt of GFT1007, for use in a method for the treatment of PBC, without provoking and/or worsening pruritus in a subject having PBC with pruritus.

The invention also relates to a pharmaceutical composition comprising a compound selected from elafibranor, GFT1007, a pharmaceutical salt of elafibranor and a pharmaceutically acceptable salt of GFT1007, for use in a method for the treatment of PBC and for the reduction in the intensity or severity of pruritus in a subject having PBC.

The invention also relates to a method for the treatment of PBC without provoking and/or worsening at least one adverse event associated to PBC in a subject in need thereof, said method comprising administering to said subject a therapeutically effective amount of elafibranor, GFT1007, or of a pharmaceutically acceptable salt of elafibranor or of GFT1007. In a particular embodiment, the adverse event is pruritus.

The invention further relates to a method for the treatment of PBC in a subject having PBC with pruritus, said method comprising administering to said subject a therapeutically effective amount of a compound selected from elafibranor, GFT1007, a pharmaceutical salt of elafibranor and a pharmaceutically acceptable salt of GFT1007.

The invention also relates to a method for the treatment of PBC in a subject having PBC with pruritus, without provoking and/or worsening pruritus, said method comprising administering to said subject a therapeutically effective amount of a compound selected from elafibranor, GFT1007, a pharmaceutical salt of elafibranor and a pharmaceutically acceptable salt of GFT1007.

The invention also relates to a method for the treatment of PBC and for the reduction in the intensity or severity of pruritus in a subject having PBC, said method comprising administering to said subject a therapeutically effective amount of a compound selected from elafibranor, GFT1007, a pharmaceutical salt of elafibranor and a pharmaceutically acceptable salt of GFT1007.

Elafibranor, GFT1007 or a pharmaceutically acceptable salt of elafibranor or of GFT1007 may be formulated in a pharmaceutical composition. In a particular embodiment, elafibranor or a pharmaceutical salt thereof is formulated in a pharmaceutical composition.

Pharmaceutical composition used in the invention can comprise one or several excipients or vehicles, acceptable within a pharmaceutical context (e.g. saline solutions, physiological solutions, isotonic solutions, etc., compatible with pharmaceutical usage and well-known by one of ordinary skill in the art). This composition can also comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. Elafibranor or GFT1007 can be formulated for enteral or parenteral administration. For example, elafibranor or GFT1007 can be formulated for oral, intravascular (e.g. intravenous or intra-arterial), intramuscular, intraperitoneal, subcutaneous, transdermal or nasal administration. The pharmaceutical composition can be a solid or liquid dosage form. Illustrative formulations include, without limitation, an injectable suspension, or suspension for oral ingestion, a gel, an oil, a pill, a tablet, a suppository, a powder, a gel cap, a capsule, an aerosol, an ointment, a cream, a patch, or means of galenic forms for a prolonged and/or slow release.

In some embodiments of the invention, GFT1007, the active metabolite of elafibranor, is used. GFT1007 is 2-[2,6-dimethyl-4-[3-[4-(methylthio)phenyl]-3-oxo-propyl]phenoxy]-2-methylpropanoic acid. Its properties and synthesis were described in PCT application WO2007/147879, where it is referred to as compound 1.

According to the present invention, the pharmaceutical composition of the invention may include a stereoisomer of elafibranor, of GFT1007, or of a pharmaceutically acceptable salt of elafibranor or of GFT1007.

A stereoisomer is an isomeric compound that has the same molecular formula and sequence of bonded atoms, but differs in the 3D-dimensional orientations of its atoms in space. The stereoisomers include enantiomers, diastereoisomers, cis-trans and E-Z isomers, conformers and tautomers.

Elafibranor or GFT1007 can be formulated as pharmaceutically acceptable salt, particularly an acid or base salt compatible with pharmaceutical use. Salts of elafibranor or GFT1007 implemented herein include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

In particular, “pharmaceutically acceptable salts” include inorganic as well as organic acids salts. Counter-ions may be selected from the following the non-exhaustive list: ammonia, L-arginine, benethamine, benzathine, tert-butylamine (erbumine), calcium hydroxide, choline hydroxide, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium hydroxide, meglumine (N-methyl-glucamine), 4-(2-hydroxyethyl)-morpholine, piperazine, potassium hydroxide, sodium hydroxide, triethanolamine (2,2′,2″-nitrilo-tris(ethanol)), tromethamine, zinc hydroxide, in particular tromethamine, potassium, sodium, benethamine, benzathine, L-arginine, ethanolamine, meglumine, glycine, erbumine, L-lysine, epolamine, choline, preferably tromethamine, potassium, sodium, benethamine, benzathine, L-arginine, more preferably tromethamine, potassium, sodium, L-arginine, more particularly tromethamine.

In particular embodiments, the invention implements an ammonia, L-arginine, benethamine, benzathine, tert-butylamine (erbumine), calcium, choline, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium, meglumine (N-methyl-glucamine), 4-(2-hydroxyethyl)-morpholine, piperazine, potassium, sodium, triethanolamine (2,2′,2″-nitrilo-tris(ethanol)), tromethamine or zinc salt of elafibranor or GFT1007. In a further particular embodiment, the salt of elafibranor or GFT1007 is selected from a tromethamine, potassium, sodium, L-arginine, benethamine, benzathine, ethanolamine, meglumine, glycine, erbumine, L-lysine, choline, epolamine, magnesium or 2-amino-2-methyl-propan-1-ol salt of elafibranor or GFT1007.

As used herein, the term “therapeutically effective amount” refers to a quantity of elafibranor or GFT1007 which prevents, removes or reduces PBC and one of its adverse events , in particular a quantity of elafibranor or GFT1007 which prevents, removes or reduces PBC and pruritus. In particular, the amount of pharmaceutical salt of elafibranor or GFT1007 is intended as the amount of free form of elafibranor or GFT1007 in this pharmaceutical salt.

The quantity to be administered can be adapted by a person skilled in the art. In particular, doses and regimen of administration may be function of the stage and of the severity of PBC and/or pruritus to be treated, as well as of the weight, the age and the global health of the subject to be treated, as well as of the judgment of the doctor.

In a particular embodiment, elafibranor, GFT1007, or a pharmaceutically acceptable salt of elafibranor or of GFT1007 is administered at a dose varying between 10 mg and 200 mg per administration, preferentially between 80 mg and 120 mg per administration. In a further particular embodiment, elafibranor, GFT1007, or a pharmaceutically acceptable salt of elafibranor or of GFT1007 is administered at a dose of 80 mg per administration. In another particular embodiment, elafibranor, GFT1007 or a pharmaceutically acceptable salt of elafibranor or of GFT1007 is administered at a dose of 120 mg per administration.

In yet another embodiment, elafibranor, GFT1007, or a pharmaceutically acceptable salt of elafibranor or of GFT1007 or a pharmaceutical composition comprising the same is administered orally. Preferably, elafibranor, GFT1007, or a pharmaceutically acceptable salt of elafibranor or of GFT1007, or a pharmaceutical composition comprising the same is orally administered once a day.

According to an embodiment, the pharmaceutical composition is a solid dosage form, such as a tablet. In a further particular embodiment, said tablet comprises between 10 mg and 200 mg of elafibranor, GFT1007, or of a pharmaceutically acceptable salt of elafibranor or of GFT1007, such as between 80 mg and 120 mg of elafibranor, GFT1007, or of a pharmaceutical salt of elafibranor or of GFT1007. For example, a tablet may comprise 80 mg of elafibranor or GFT1007 or a pharmaceutical salt of elafibranor or of GFT1007 or 120 mg of elafibranor or GFT1007 or a pharmaceutical salt of elafibranor or of GFT1007.

In yet another embodiment, a tablet comprising 80 mg of elafibranor or GFT1007 is orally administered once a day.

In yet another embodiment, a tablet comprising 120 mg of elafibranor or GFT1007 is orally administered once a day.

In a particular variant of all the embodiments described above, the compound is elafibranor or a pharmaceutically acceptable salt thereof. In yet another particular variant of all the embodiments described above, the compound is elafibranor.

### LEGENDS OF THE FIGURES

FIG. 1: Relative Change from Baseline in Serum Alkaline Phosphatase at Endpoint—

Primary Efficacy Endpoint—Primary and Supportive Analyses

ALP=alkaline phosphatase; ANCOVA=analysis of covariance; CI=confidence interval; EOT=end-of-treatment; SD=standard deviation; trt=treatment.

^{a}Non-parametric randomization-based ANCOVA with baseline ALP as a covariate. p-values were computed under the null hypothesis (based on re-randomizations of the population) while estimates and CIs were computed under the alternative hypothesis (based on repeated random sampling).

^{b}ANCOVA with baseline ALP as covariate and without interaction term.

FIG. 2: Mean Alkaline Phosphatase Values from Baseline through Week 12 by Treatment Group

FIG. 3: Mean Relative Change from Baseline through Week 12 in Alkaline Phosphatase by Treatment Group

FIG. 4: Absolute and Relative Change from Baseline in Pruritus Visual Analogue Score

EOT=end-of-treatment; SD=standard deviation; VAS=visual analogue scale.

FIG. 5: Median Relative Change from Baseline through Week 12 in Pruritus VAS

FIG. 6: Absolute and Relative Change from Baseline to Endpoint in Pruritus Domain of the PBC-40 Quality of Life Questionnaire

EOT=end-of-treatment;

SD=standard deviation;

VAS=visual analogue scale.

### EXAMPLES

### Example 1: Enrollment

The study was a Phase 2, double-blind, randomized, parallel group, placebo-controlled study, designed to evaluate the efficacy and safety of elafibranor at doses of 80 mg or 120 mg daily vs placebo in an adult PBC population. This study projected to randomize approximately 45 subjects to 3 treatment groups in a 1:1:1 ratio (elafibranor 80 mg, elafibranor 120 mg, or placebo).

In order to participate in the study, all subjects must have met all of the following criteria:
Was a male or female between 18 to 75 years of age
Had a definite or probable PBC diagnosis demonstrated by the presence of at least 2 of the following 3 diagnostic factors:
History of elevated ALP levels for at least 6 months
Positive antimitochondrial Ab titers (>1/40 on immunofluorescence or antiinflammatory macrophages positive by enzyme-linked immunosorbent assay) or positive PBC-specific antinuclear antibodies
Liver biopsy consistent with PBC
Had an ALP≥1.67×(Upper Limit of Normal—104 U/L for women)
Received UDCA for at least 12 months (stable dose for ≥6 months) before the screening visit.

Moreover, in order to have been eligible for enrollment in the study, none of the following criteria could have applied to any subject:
Had a history or presence of other concomitant liver diseases including:
Positive hepatitis B surface antigen (HBsAg) at Screening
Positive hepatitis C virus (HCV) RNA
Alcoholic liver disease
Primary sclerosing cholangitis
Definite autoimmune hepatitis (AIH), or “AIH-PBC overlap syndrome”
Biopsy confirmed non-alcoholic steatohepatitis
Known history of alpha-1 antitrypsin deficiency or other metabolic forms of
chronic liver disease
Gilbert's Syndrome

Subject participation was planned to be a maximum of 20 weeks. The study was comprised of 3 periods: a Screening Period, a Treatment Period, and a Follow-up Period. The Screening Period (Week −4 to Week −1) preceded the 12-week double-blind Treatment Period.

The mean age at study entry was 59.1 years, with a minimum of 40 and a maximum of 74 years. Gender was disproportionate, with 95.6% females. The majority of subjects were white (97.8%) and not Hispanic or Latino (77.8%). The mean BMI at baseline was 26.9 kg/m2 (minimum 19.1 kg/m2, maximum 39.7 kg/m2). Among female subjects, 11.1% were of child bearing potential; the remaining female subjects were postmenopausal (78.9%) or surgically sterile (21.1%). The 3 treatment groups had similar demographic and baseline characteristics, which were representative of the clinical PBC population.

### Example 2: Endpoints

The tolerability and safety of once-per-day oral administration of elafibranor in 80-mg and 120-mg doses in subjects with PBC was assessed as follows:
Physical examination
Vital signs
Medical history
ECG
Hematological parameters
Liver markers
Other biochemical safety parameters

A decrease in ALP levels is recognized as a particularly relevant surrogate marker for the treatment of PBC. Consequently the primary endpoint of the study is to evaluate the efficacy of elafibranor 80 mg or 120 mg with respect to relative change from baseline in serum ALP levels compared to placebo.

Considering that pruritus is a preeminent adverse event in subjects with PBC, assessment of pruritus was also considered. More specifically, the change from baseline in pruritus (through 5D-itch scale (measuring the degree, duration, direction [improvement or worsening], disability [effect on daily activities], and distribution of itching) and visual analogue scale for pruritus (VAS).

The 5D-itch scale is a reliable, multidimensional measure of itching that has been validated in patients with chronic pruritus to detect changes over time (Elman S, et al., The 5-D itch scale: a new measure of pruritus. Br J Dermatol. 2010; 162(3):587-593.). The VAS is a reliable and validated method of pruritus assessment (Reich A, et al., Visual analogue scale: evaluation of the instrument for the assessment of pruritus. Acta Derm Venereol.2012; 92(5):497-501).

### Example 3: Drug Used

Elafibranor (2-(2,6-dimethyl-4-{3-[4-(methylsulfanyl)phenyl]-3-oxopropen-1-yl}phenoxy)-2-methylpropanoic acid) was supplied as 80 mg or 120 mg white to offwhite round coated tablets with no printed inscription.

Two placebo tablets (each one of the same size as the corresponding active tablet) to match elafibranor 80 mg or 120 mg were provided as a white to off-white round coated tablet with no printed inscription. The placebo tablets contained the same excipients as the active formulation as well as lactose monohydrate (which was used in place of the active ingredient).

### Example 4: Results on ALP Levels

The mean relative change (%) from baseline to Endpoint in serum ALP was −48.3% for the elafibranor 80 mg treatment group, −40.6% for the elafibranor 120 mg treatment group, and 3.2% for placebo.

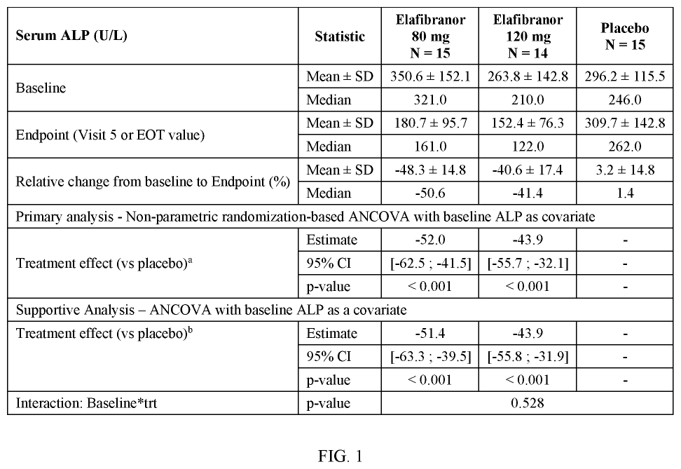

In the primary efficacy analysis conducted using a non-parametric randomization-based ANCOVA with baseline ALP as covariate, each dose demonstrated a statistically significant treatment effect vs placebo (p<0.001). The treatment effect estimate was −52.0% (95% CI [−62.5; −41.5]) for the elafibranor 80 mg treatment group and −43.9% (95% CI [−55.7; −32.1]) for the elafibranor 120 mg treatment group (FIG. 1).

The primary efficacy supportive analysis conducted using an ANCOVA with baseline ALP as a covariate was consistent with the primary efficacy analysis. The treatment effect estimate was −51.4% (95% CI [−63.3; −39.5]) for the elafibranor 80 mg treatment group and −43.9% (95% CI [−55.8; −31.9]) for the elafibranor 120 mg treatment group (FIG. 1).

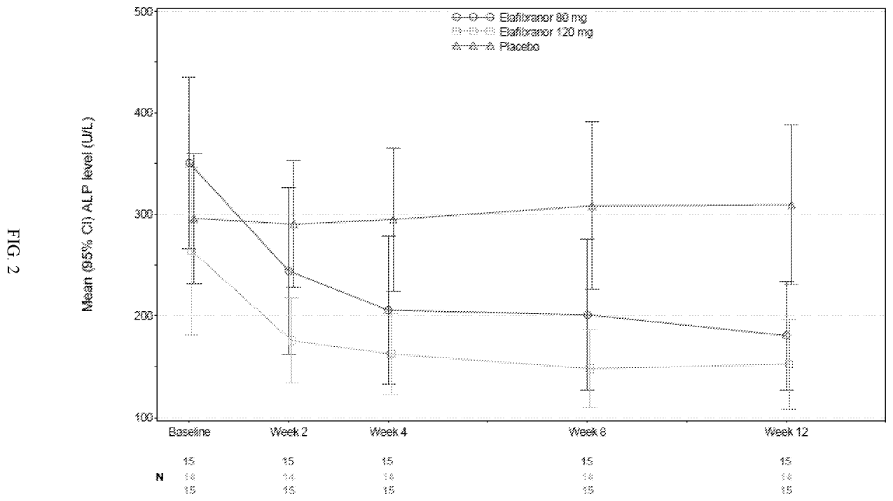

The mean (95% CI) ALP values from baseline through Week 12 are shown in FIG. 2 by treatment group. Both the elafibranor 80 mg and 120 mg treatment groups demonstrated declining mean ALP values over the 12 week study.

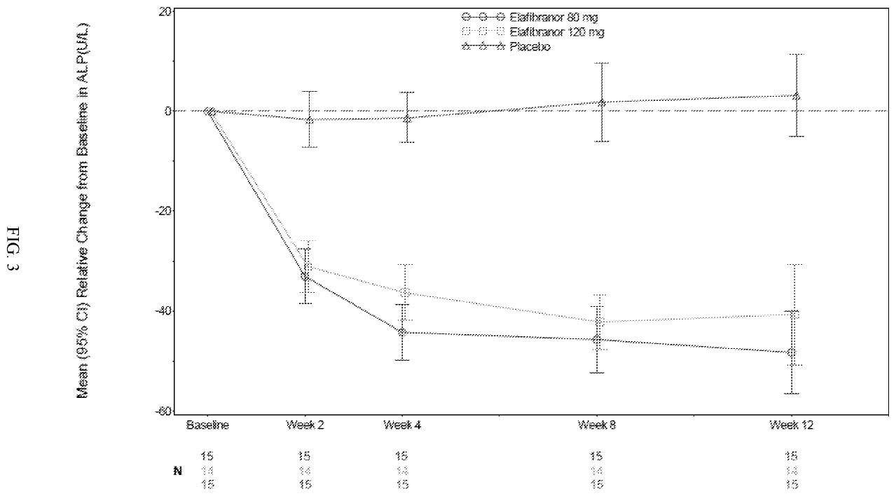

The mean (95% CI) relative changes (%) in ALP values from baseline through Week 12 are shown in FIG. 3 by treatment group. The mean relative changes (%) from baseline shows a decrease in ALP values over time for the elafibranor 80 mg and 120 mg treatment groups beginning at Week 2 and continuing up through Week 12.

The relative change from baseline in serum ALP was statistically significantly different from placebo at Endpoint for both the elafibranor 80 mg treatment group and the elafibranor 120 mg treatment group.

### Example 5: Change in Pruritus (Through 5D-Itch Scale and VAS)

Pruritus scoring values and absolute changes from baseline are provided for each domain of the 5D-itch scale (duration, degree, direction, disability, and distribution) and for the VAS by subject and by visit; baseline and Endpoint values are flagged in this listing; total scores at each visit are also summarized in this listing.

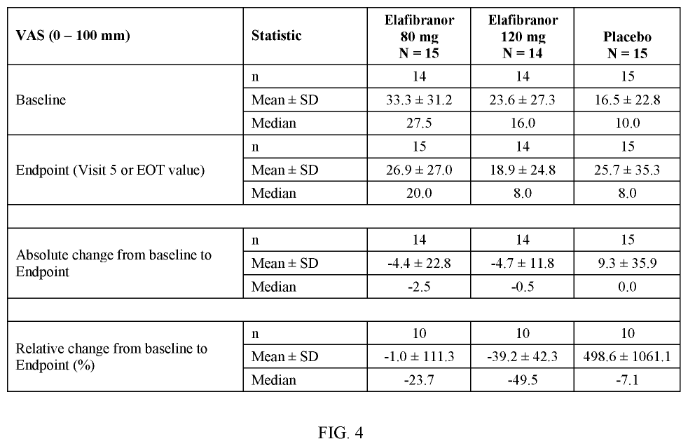

Summaries of the pruritus scoring values, absolute changes in pruritus scoring values, and relative changes in pruritus scoring values for the 5D-itch scale (duration, degree, direction, disability, and distribution) and for the VAS were assessed. The median relative change from baseline to Endpoint in the pruritus VAS were −23.7%, −49.5%, and −7.1% for the elafibranor 80 mg, elafibranor 120 mg, and placebo treatment groups, respectively (FIG. 4).

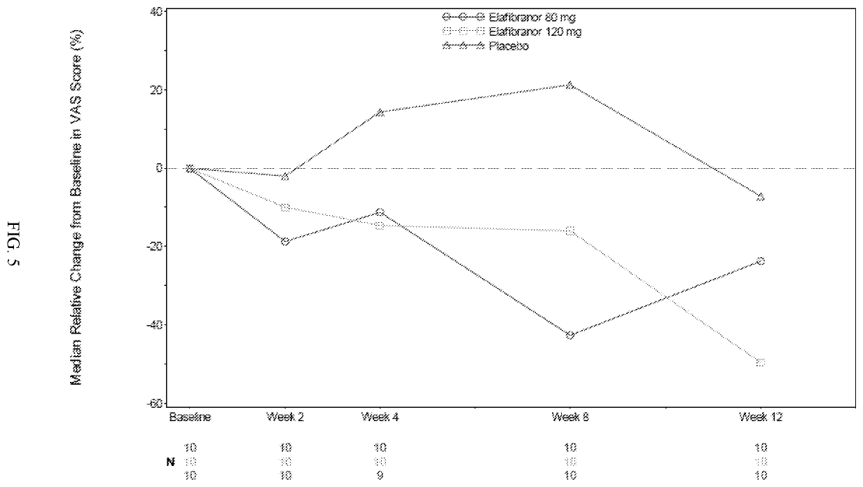

The median relative changes (%) in the pruritus VAS from baseline through Week 12 are shown in FIG. 5 by treatment group. Both the elafibranor 80 mg and the elafibranor 120 mg treatment groups demonstrated declining median VAS as early as Week 2.

### Example 6: Absolute and Relative Change from Baseline in Quality of Life (Using PBC-40 Questionnaire)

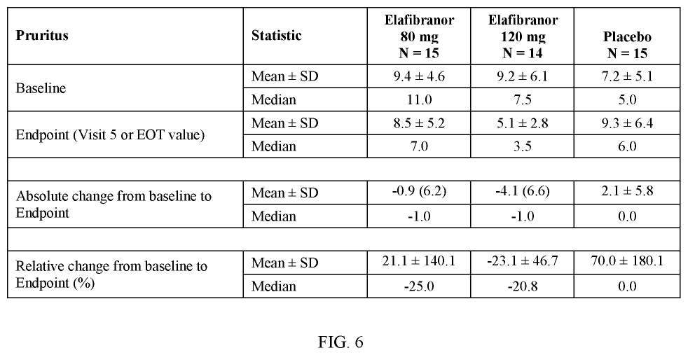

Mean absolute changes from baseline to Endpoint in the pruritus (itching) domain were −0.9, −4.1, and 2.1 for the elafibranor 80 mg, the elafibranor 120 mg, and the placebo treatment groups, respectively. Median absolute changes from baseline to Endpoint in the pruritus/itching domain were −1.0, −1.0, and 0.0 for the elafibranor 80 mg, the elafibranor 120 mg, and the placebo treatment groups, respectively. Median relative changes from baseline to Endpoint in the pruritus/itching domain were −25.0%, −20.8%, and 0% for the elafibranor 80 mg, the elafibranor 120 mg, and the placebo treatment groups, respectively. (FIG. 6).

### CONCLUSION

The percentage of subjects reporting TEAEs (Treatment-Emergent Adverse Events) was 80.0%, 86.7%, and 80.0% for the elafibranor 80 mg, elafibranor 120 mg, and placebo groups, respectively. There was no worsening of pruritus with elafibranor. On the contrary, a higher relative reduction from baseline to Endpoint in the pruritus VAS scoring values was demonstrated for both elafibranor 80 mg (−23.7%) and 120 mg treatment groups (−49.5%) compared to the placebo (−7.1%) treatment group.

This reduction in the intensity/severity of pruritus was associated with an improvement in terms of quality of life as measured by the median relative change from baseline to Endpoint in the pruritus domain of PBC 40-questionnaire.

1. A method of treating primary biliary cholangitis (PBC) without provoking and/or worsening at least one adverse event associated with PBC, the method comprising administering to a subject in need thereof a pharmaceutical composition comprising a compound selected from 2-(2,6-dimethyl-4-{3-[4-(methylsulfanyl)phenyl]-3-oxopropen-1-yl}phenoxy)-2-methylpropanoic acid (elafibranor), 2-[2,6-dimethyl-4-[3-[4-(methylthio)phenyl]-3-oxo-propyl]phenoxy]-2-methylpropanoic acid (GFT1007), a pharmaceutically acceptable salt of elafibranor, and a pharmaceutically acceptable salt of GFT1007.

2. The method according to claim 1, wherein the adverse event is pruritus.

3. A method of treating primary biliary cholangitis (PBC), the method comprising administering to a subject having PBC with pruritus a pharmaceutical composition comprising a compound selected from 2-(2,6-dimethyl-4-{3-[4-(methylsulfanyl)phenyl]-3-oxopropen-1-yl}phenoxy)-2-methylpropanoic acid (elafibranor), 2-[2,6-dimethyl-4-[3-[4-(methylthio)phenyl]-3-oxo-propyl]phenoxy]-2-methylpropanoic acid (GFT1007), a pharmaceutically acceptable salt of elafibranor, and a pharmaceutically acceptable salt of GFT1007.

4. The method according to claim 3, wherein the treatment does not provoke and/or worsen the pruritus.

5. The method according to claim 1, wherein said composition is a tablet, injectable suspension, gel, oil, pill, suppository, powder, gel cap, capsule, aerosol, or a prolonged and/or slow release dosage form.

6. The method according to claim 1, wherein said composition is orally administered once a day.

7. The method according to claim 1, wherein elafibranor, GFT1007, or the pharmaceutically acceptable salt of elafibranor or of GFT1007 is administered at a dose varying between 10 mg and 200 mg per administration.

8. The method according to claim 1, wherein elafibranor, GFT1007, or the pharmaceutically acceptable salt of elafibranor or of GFT1007 is administered at a dose varying between 80 mg and 120 mg per administration.

9. The method according to claim 1, wherein said pharmaceutical composition is a tablet comprising 80 mg of elafibranor or of GFT1007.

10. The method according to claim 1, wherein said pharmaceutical composition is a tablet comprising 120 mg of elafibranor or of GFT1007.

11. The method according to claim 1, wherein the compound is elafibranor or the pharmaceutically acceptable salt thereof.

12. The method according to claim 1, wherein the compound is elafibranor.

13. The method according to claim 3, wherein said composition is a tablet, injectable suspension, gel, oil, pill, suppository, powder, gel cap, capsule, aerosol, or a prolonged and/or slow release dosage form.

14. The method according to claim 3, wherein said composition is orally administered once a day.

15. The method according to claim 3, wherein elafibranor, GFT1007, or the pharmaceutically acceptable salt of elafibranor or of GFT1007 is administered at a dose varying between 10 mg and 200 mg per administration.

16. The method according to claim 3, wherein elafibranor, GFT1007, or the pharmaceutically acceptable salt of elafibranor or of GFT1007 is administered at a dose varying between 80 mg and 120 mg per administration.

17. The method according to claim 3, wherein said pharmaceutical composition is a tablet comprising 80 mg of elafibranor or of GFT1007.

18. The method according to claim 3, wherein said pharmaceutical composition is a tablet comprising 120 mg of elafibranor or of GFT1007.

19. The method according to claim 3, wherein the compound is elafibranor or the pharmaceutically acceptable salt thereof.

20. The method according to claim 3, wherein the compound is elafibranor.
